(19) United States
(12) Patent Application Publication
(10) Pub. No.: US 2019/0107508 A1
(43) Pub. Date: Apr. 11, 2019

(54) SENSOR, METHOD OF FORMING A SENSOR AND USE THEREOF

(71) Applicant: Cambridge Display Technology Ltd., Godmanchester (GB)

(72) Inventors: Irene Zaccari, Godmanchester (GB); Jonathan Behrendt, Godmanchester (GB)

(73) Assignee: Cambridge Display Technology Ltd., Godmanchester (GB)

(21) Appl. No.: 16/087,904

(22) PCT Filed: Feb. 27, 2017
(86) PCT No.: PCT/GB2017/050521

§ 371 Date: Sep. 24, 2018

(30) Foreign Application Priority Data
Mar. 31, 2016 (GB) 1605492.6

(51) Int. Cl. G01N 27/414; G01N 33/543; H01L 51/05

(52) U.S. Cl. CPC G01N 27/4145; H01L 51/0545; G01N 33/5438

(57) ABSTRACT

A sensor for detection of a biological analyte comprises: a bottom gate thin film transistor comprising a gate electrode (103); source and drain electrodes (107, 109); a dielectric layer (105) between the gate electrode and the source and drain electrodes; and a semiconductor layer (111) comprising an organic semiconducting material extending between the source and drain electrodes; a binding layer comprising an amphiphilic polymer comprising a binding group (115) adjacent to and in direct contact with the semiconductor layer; and a receptor (220) bound to the binding group.

### FIELD OF THE INVENTION

The invention relates to sensors, in particular biosensors, use of said sensors and methods of forming said sensors.

### BACKGROUND OF THE INVENTION

A wide range of sensors for detection of an analyte are known.

US 2013/0071289 discloses a sensor comprising a transistor having a coupling/stabilization layer covering at least part of the semiconductor layer of the transistor.

Katz et al, “Label-free brain injury biomarker detection based on highly sensitive large area organic thin film transistor with hybrid coupling layer”, Chem. Sci., 2014, 5, 416 discloses an OTFT biosensor for sensing of glial fibrillary acidic protein (GFAP) having a CYTOP passivation layer, a vapour-deposited layer of tetratetracontane (C₄₄H₉₀) to fill residual pinholes in CYTOP layer and a layer of PS-block-PAA polymer having anti-GFAP immobilized thereon.

Kergoat, et al. “DNA detection with a water-gated organic field-effect transistor” Org. Electron. 2012, 13, 1-6 discloses a water-gated organic field-effect transistor poly [3-(5-carboxypentyl)thiophene-2,5-diyl] onto which DNA probes are covalently grafted via NHS/EDC chemistry.

It is an object of the invention to provide a sensor providing an electrical response indicating presence of a target analyte.

It is a further object of the invention to provide a sensor having a simple structure.

### SUMMARY OF THE INVENTION

In a first aspect the invention provides a sensor comprising:

a bottom gate thin film transistor comprising a gate electrode; source and drain electrodes; a dielectric layer between the gate electrode and the source and drain electrodes; and a semiconductor layer comprising an organic semiconducting material extending between the source and drain electrodes;
a binding layer comprising an amphiphilic polymer comprising a binding group adjacent to and in direct contact with the semiconductor layer; and
a receptor bound to the binding group.

In a second aspect, the invention provides a method of forming a sensor according to the first aspect, the method comprising the step of depositing a solution or suspension comprising the amphiphilic polymer onto the semiconductor layer. The invention describes how to functionalise a surface which typically has no functional groups and can readily be damaged by treatment with organic solvents (i. e. the surface of a film of OSC). The method that we have disclosed specifically uses non-covalent absorption (rather than covalent attachment) of amphiphilic polymers.

In a third aspect the invention provides a sensor comprising:

a bottom gate thin film transistor comprising a gate electrode; source and drain electrodes; a dielectric layer between the gate electrode and the source and drain electrodes; and a semiconductor layer comprising an organic semiconducting material extending between the source and drain electrodes;
a binding layer comprising a protein comprising a binding material adjacent to and in direct contact with the semiconductor layer; and
a receptor bound to the binding group.

The invention also provides a method for detecting a target analyte, the method comprising the step of contacting a sensor according to the first aspect or the third aspect with an analyte fluid and measuring a response from the sensor.

### DESCRIPTION OF THE DRAWINGS

FIG. 1 illustrates a bottom-gate OTFT having a binding layer thereon that is suitable for forming a sensor according to an embodiment of the invention;

FIG. 2A illustrates formation of a sensor according to an embodiment of the invention by binding a receptor to binding groups of the binding layer of the device of FIG. 1;

FIG. 2B illustrates use of the sensor according to FIG. 2A to detect a target analyte;

FIG. 3A illustrates the device of FIG. 1 wherein a linking material is bound to binding groups of the binding layer of the device of FIG. 1;

FIG. 3B illustrates formation of a sensor according to an embodiment of the invention by binding a receptor to the linking material;

FIG. 3C illustrates use of the sensor of FIG. 3B to detect a target analyte;

FIG. 4 is a graph showing effect on current for a bottom gate OTFT device having a binding layer thereon upon exposure of the device to a solution comprising a receptor and to a buffer solution; and

FIG. 5 is a graph showing effect on current for a sensor according to an embodiment of the invention upon exposure of the sensor to an analyte solution and to a buffer solution.

### DETAILED DESCRIPTION OF THE INVENTION

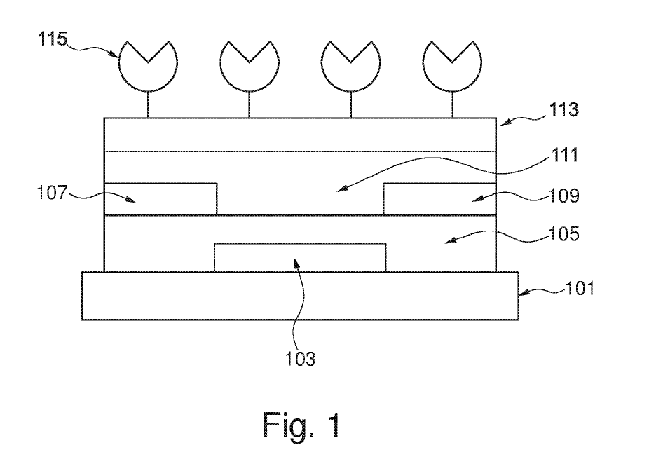

FIG. 1, which is not drawn to any scale, illustrates a bottom gate organic thin film transistor 100 for forming a detector of a sensor, preferably a biosensor, according to an embodiment of the invention.

The bottom-gate OTFT comprises a gate electrode 103 over a substrate 101; source and drain electrodes 107, 109; a dielectric layer 105 between the gate electrode and the source and drain electrodes; and a semiconductor layer 111 comprising or consisting of an organic semiconducting material and extending between the source and drain electrodes. The bottom-gate OTFT may be a n-type or p-type device.

The bottom-gate OTFT may consist of the layers described with reference to FIG. 1 or it may comprise one or more further layers. Exemplary further layers include, without limitation, a charge-transporting layer between the source and drain electrodes and the semiconductor layer, for example as described in WO2016/001095; and more than one layer between the source and drain electrodes and the gate electrode, for example as described in US 2011/127504.

A binding layer 113 is provided adjacent to and in contact with the semiconductor layer 111. The binding layer comprises or consists of a binding material comprising a binding group 115. A receptor or a linking group as described herein may be bound to the binding layer by specific binding to the binding group as described herein. Additionally, the binding layer may prevent non-specific binding of components of an analyte fluid as described herein.

The binding material is preferably an amphiphilic polymer comprising a binding group. The binding layer is preferably more wettable by an analyte liquid than the organic semiconducting layer. An increase in wettability may be determined by a reduction in contact angle of the analyte fluid on a surface. Preferably, the binding layer has a contact angle with water of less than 90°.

One of the hydrophilic and hydrophobic parts of the amphiphilic polymer may adhere to the organic semiconducting layer to form a binding layer that is stable under conditions used in use of the sensor. Optionally, at least 90 wt %, preferably substantially all, of the amphiphilic polymer of the binding layer of the sensor remains upon washing by water at 25° C. for 1 hour.

Amphiphilic polymers as described herein may be a synthetic or naturally occurring polymer comprising a binding group.

Synthetic polymers include homopolymers and polymers comprising two or more different repeat units. Exemplary synthetic polymers comprise vinylalcohol or vinylpyrollidone repeat units.

A synthetic polymer may be substituted with hydrophobic and hydrophilic substituents, optionally non-polar substituents such as hydrocarbyl groups and polar substituents such as polyether groups. Exemplary hydrocarbyl groups are C₁₋₂₀ alkyl groups; unsubstituted C₅₋₂₀ aryl groups; and C₅₋₂₀ aryl groups substituted with one or more C₁₋₁₂ alkyl groups.

The polymer may be a block copolymer comprising at least one hydrophobic block and at least one hydrophilic block.

The binding material is preferably a protein, optionally bovine serum albumin (BSA), casein or gelatin, comprising a binding group.

The binding group is optionally a thiol group, a maleimide group, a streptavidin group or a biotin group.

Preferably, the binding material is BSA comprising a binding group, more preferably biotinylated BSA or maleimide-activated BSA.

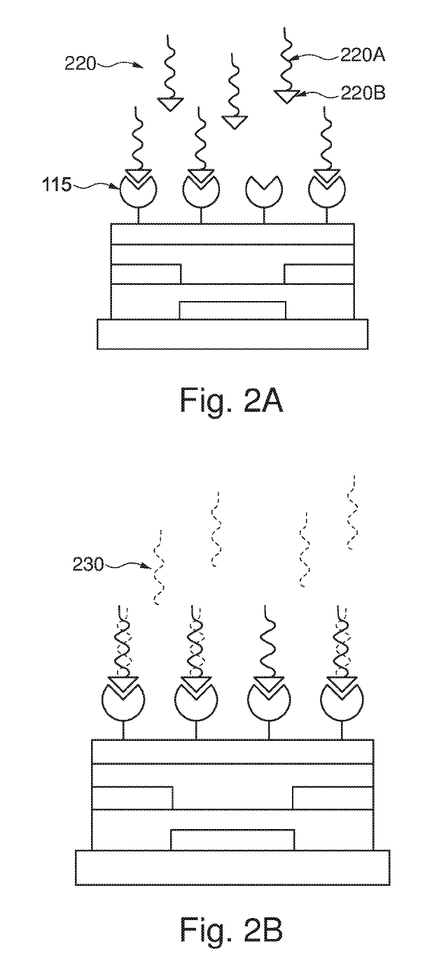

With reference to FIG. 2A, a receptor 220 may be bound directly to the binding group 115 to form the sensor. In FIG. 2A the receptor is DNA 220A modified with a receptor binding group 220B to bind to the binding group 115 although it will be appreciated that other receptors may be used.

Optionally, the binding material of the binding layer 113 of this embodiment is maleimide-activated BSA and the receptor binding group 220B is thiol.

In use, the sensor binds to complementary DNA analyte strands 230 in an analyte fluid as shown in FIG. 2B. In use the sensor is exposed to an analyte fluid, preferably an analyte liquid. If a target analyte 301 is present within the analyte fluid then it may bind to the receptors. Binding of the target analyte to the receptors may cause a change in a measurable property of the sensor, enabling detection of the target analyte and/or the concentration thereof. The measurable property of the sensor may be its threshold voltage (V_{T}) or drain current (Id). Accordingly, the sensor may be used for real time and/or label-free sensing of analytes in an analyte fluid.

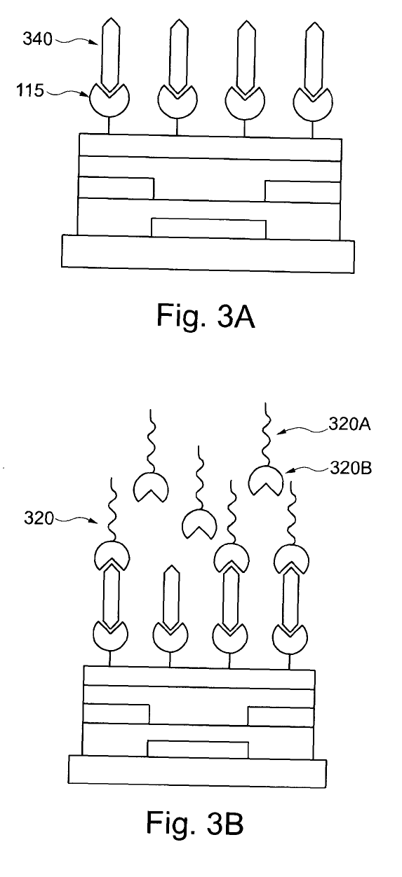

The sensor described with reference to FIGS. 2A and 2B comprises receptors bound directly to binding groups 115 of the binding layer 113. FIGS. 3A-3C illustrated an embodiment in which receptors are not bound directly to the binding groups 115.

With reference to FIG. 3A, a linking material 340 is brought into contact with the binding layer 113 and binds to the binding group 115. With reference to FIG. 3B, the sensor is formed by binding receptor 320 to the linking material 340. In FIG. 3B the receptor 320 is DNA 320A modified with a receptor binding group 320B to bind to linking material 340, although it will be appreciated that other receptors may be used. In use, the sensor binds to complementary DNA analyte strands 330, as shown in FIG. 3C.

Optionally, the binding material is biotinylated BSA, the linking material is streptavidin and the receptor comprises biotin.

A wide range of receptors are known to the skilled person. A receptor as described herein is preferably a biomolecule. Exemplary biomolecules include, without limitation:

biological material, optionally peptides, carbohydrates, antibodies, antigens, enzymes, proteins, cell receptors, DNA, RNA, PNA, aptamers and natural products;
biologically derived material, optionally recombinant antibodies, engineered proteins; and
biomimics, optionally synthetic receptors, biomimetic catalysts, combinatorial ligands and imprinted polymers.

The receptor may be modified, if needed, to form a receptor binding group for binding to a linking material or a binding group of the binding layer.

The binding layer is formed by depositing a solution or suspension comprising the binding material dissolved or suspended in at least one solvent on the semiconductor layer. The solution or suspension may consist of the at least one solvent and the binding material or may comprise one or more further materials dissolved or suspended in the solvent or solvents. One of the hydrophobic or hydrophilic part of the amphiphilic polymer may adhere to the organic semiconducting layer. Optionally, excess amphiphilic polymer may be removed by washing to leave the amphiphilic polymer of the binding layer.

Preferably, the solvent is water. The solution may be an aqueous buffer solution, optionally a sodium phosphate buffer. Preferably, the solution of the binding material has a pH in the range of 6-8. Washing may be done with a buffer solution.

The linking material, if present, and the receptor may be deposited by any method.

The linking material, if present, and the receptor may each be deposited from a solution or suspension in at least one solvent. Preferably, the solvent is water. The solution may be an aqueous buffer solution, optionally a sodium phosphate buffer. Preferably, the solution has a pH in the range of 6-8.

Preferably, the pH of a buffer solution used to deposit the linking material is close to the isoelectric point of the binding material in solution, optionally within a pH of 0.5 or less. In the case of a charged linking material, the buffer pH may be between the isoelectric points of the binding material and the linking material.

Preferably, the pH of the buffer solution used to deposit the receptor is close to the isoelectric point of the material it binds to in solution, i.e. the binding material or the linking material, optionally within a pH of 0.5 or less. In the case of a charged receptor, the buffer pH may be between the isoelectric points of the receptor and the material it binds to.

The binding layer may have a thickness in the range of 1-50 nm, optionally 4-20 nm.

Exemplary analyte fluids include, without limitation: human or animal bodily fluids, optionally a liquid selected from blood, urine, saliva, tears, faeces, gastric fluid, bile, sweat, cerebrospinal fluid and amniotic fluid; cell culture media or other biological samples; food; environmental water, e.g. river, sea or rain water; wine; soil extracts; and gases or other non-biological samples. Exemplary target analytes include, without limitation, DNA, RNA, peptides, carbohydrates, antibodies, antigens, enzymes, proteins, hormones, bacteria, viruses, protozoa, and small molecules, both synthetic and biological. Preferably, the target analyte is a positively or negatively charged material. Preferably, the target analyte is one or more of bacteria, viruses, protozoa or biomarker.

The term ‘small molecules’ as used herein means, in the context of the biological sciences and pharmacology, low molecular weight (typically <900 daltons) compounds that are biologically active. In chemical biology, the term also covers metal ions. Small molecules include, without limitation, lipids, monosaccharides, second messengers and metabolites, as well as drugs and xenobiotics.

### Organic Thin Film Transistor

The semiconductor layer of the organic thin film transistor may comprise or consist of any organic semiconducting material including polymeric and non-polymeric materials. The semiconductor layer may comprise a blend of a non-polymeric organic semiconductor and a polymer, optionally a conjugated polymer. Exemplary organic semiconductors are disclosed in WO 2016/001095, the contents of which are incorporated herein by reference.

The semiconducting layer may be deposited by any suitable technique, including evaporation and deposition from a solution comprising or consisting of one or more organic semiconducting materials and at least one solvent. Exemplary solvents include mono- or poly-alkylbenzenes such as toluene and xylene; tetralin; and chloroform. The solvent is preferably a non-polar solvent, optionally benzene substituted with one or more C₁₋₁₀ alkyl or alkoxy groups. Solution deposition techniques include coating and printing methods, for example spin coating dip-coating, ink jet printing, roll printing and screen printing.

The organic semiconductor layer may be hydrophobic. The organic semiconductor layer may have a contact angle with water of greater than 90°. The hydrophobicity of the organic semiconducting layer may be affected by the structure of the material or materials of the layer and any substituents thereof.

The organic semiconductor is preferably substituted with non-polar groups, optionally hydrocarbyl groups. Exemplary hydrocarbyl groups are C₁₋₂₀ alkyl groups; unsubstituted C₅₋₂₀ aryl groups; and C₅₋₂₀ aryl groups substituted with one or more C₁₋₁₂ alkyl groups. Any other components of the organic semiconductor layer are optionally substituted with hydrocarbyl groups as described herein.

Use of an amphiphilic binding material with a hydrophobic organic semiconductor layer may enhance wettability of aqueous analyte solutions.

The length of the channel defined between the source and drain electrodes may be up to 500 microns, but preferably the length is less than 200 microns, more preferably less than 100 microns, most preferably less than 20 microns.

The semiconducting layer extends between the source and drain electrodes and may extend over the source and/or drain electrodes.

The gate electrode can be selected from a wide range of conducting materials for example a metal (e.g. gold) or metal compound (e.g. indium tin oxide). Alternatively, conductive polymers may be deposited as the gate electrode. Such conductive polymers may be deposited from solution using, for example, spin coating or ink jet printing techniques and other solution deposition techniques discussed above.

The insulating layer comprises a dielectric material. Preferably, the dielectric constant, k, of the dielectric is at least 2 or at least 3. The dielectric material may be organic or inorganic. Preferred inorganic materials include metal oxides, SiO2, SiNx and spin-on-glass (SOG). Preferred organic materials are polymers and include insulating polymers such as poly vinylalcohol (PVA), polyvinylpyrrolidine (PVP), acrylates such as polymethylmethacrylate (PMMA) and benzocyclobutanes (BCBs). The insulating layer may be formed from a blend of materials or comprise a multi-layered structure.

The dielectric material may be deposited by thermal evaporation, vacuum processing or lamination techniques as are known in the art. Alternatively, the dielectric material may be deposited from solution using, for example, spin coating or ink jet printing techniques and other solution deposition techniques discussed above. If the dielectric material is deposited from solution then the dielectric material should not be dissolved if an organic semiconductor is deposited onto it from solution. Techniques to avoid such dissolution include: use of orthogonal solvents for example use of a solvent for deposition of the organic semiconducting layer that does not dissolve the dielectric layer; and cross linking of the dielectric layer before deposition of the organic semiconductor layer. The thickness of the insulating layer is preferably less than 500 nm, more preferably in the range of 1-200 nm, optionally 5-50 nm.

For ease of manufacture the source and drain electrodes are preferably the same material, preferably a metal which may be deposited by any suitable method, optionally thermal evaporation. The OTFT preferably has an operational voltage of less than 5V, optionally less than 3V or less than 1V, for example as disclosed in J. Am. Chem. Soc., 2005, 127 (29), pp 10388-10395, ACS Appl. Mater. Interfaces, 2011, 3 (12), pp 4662-4667 and Organic Electronics Volume 17, February 2015, Pages 178-183, the contents of which are incorporated herein by reference.

The sensor has been described herein with reference to a sensor in which the detector is a bottom gate OTFT. It will be appreciated that other detectors which provide an electrical response upon binding of a target analyte to receptors may be used in place of a bottom gate OTFT in a sensor as described herein including, without limitation, extended gate or floating gate organic field effect transistors and electrolytically gated field effect transistors, for example as disclosed in Organic Electronics 2012; 13(1):1-6, the contents of which are incorporated herein by reference.

### Applications

Applications of the sensor as described herein include, without limitation: pathogen detection, diagnostics, detection of disease related biomarkers, environmental monitoring, food safety control and military purposes, for example:
Glucose monitoring in diabetes patients
Detection of pesticides and river water contaminants
Remote sensing of airborne bacteria e.g. in counter-bioterrorist activities
Determining levels of toxic substances before and after bioremediation
Detection of organophosphate
Detection of electron acceptors, e.g. trinitrotoluene
Measurement of folic acid or vitamin B12
Determination of drug residues in food, such as antibiotics and growth promoters, particularly meat and honey
Drug discovery and evaluation of biological activity of compounds
Detection of toxic metabolites such as mycotoxins

### EXAMPLES

A bottom gate organic thin film transistor was formed wherein the organic semiconducting layer was formed by spin-coating a layer of the polymer DTBTM-BT from xylene:

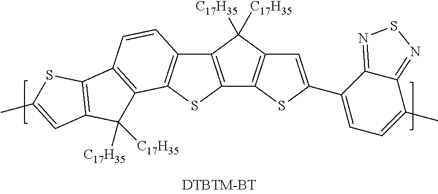

Biotin-modified BSA, supplied by Thermo Fisher Scientific, was deposited on the organic semiconducting layer by depositing a droplet of a solution containing the biotin-modified BSA at a concentration of 50 ug/ml in 100 mM phosphate buffer pH 7.4 on the organic semiconducting layer and leaving at room temperature for 2 hours (30 min-16 h). The treated area was then washed 3 times with the same buffer.

Streptavidin was bound to the biotin-BSA by depositing a droplet of a solution of 50 ug/ml of streptavidin in 100 mM Acetate buffer pH 5.5 for between 15 min to 2 h on the biotin-modified BSA. The treated area was then washed 3 times with the same buffer.

To complete the sensor biotin-DNA, supplied by Thermo Fisher Scientific, was immobilised on the surface by depositing a droplet of a solution of Biotin-DNA at a concentration of 20 uM in 100 mM Acetate buffer pH 5.5 on the modified OSC for 1 hour and leaving at room temperature for 1 h. The treated area was then washed three times with the same buffer.

Alternatively, 400 ul of 20 uM biotin-DNA in 10 mM phosphate buffer (PB) pH 7 was flushed in a flow chamber over the streptavidin and left incubated while measuring.

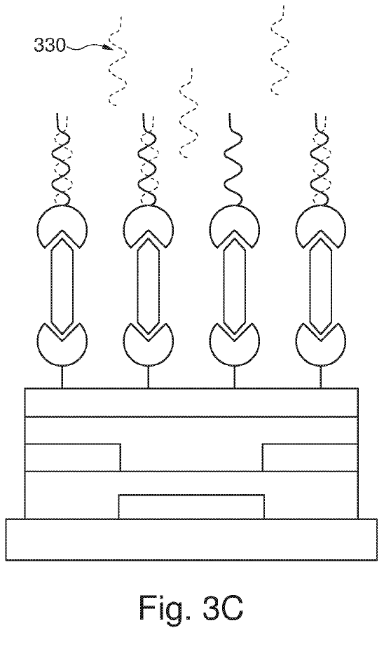

The measurement was performed by fixing the Vgs at −1V (between Vth and −1.5 V) and Vds at −1V (between −0.1 V and −1.5 V) and recording the gate, source and drain currents every 15 seconds. The binding of the DNA target was measured in the same conditions. 400 ul 1 uM DNA solution in 10 mM PB pH 7 was in a flow chamber and left incubating while measuring. With reference to FIG. 4, no significant change in drain current is observed upon exposure of the streptavidin to a buffer solution containing no biomolecules, however a significant drop in drain current is observed upon exposure to the buffer solution with biotinylated DNA dissolved therein, demonstrating a sensitivity of the OTFT to binding of DNA.

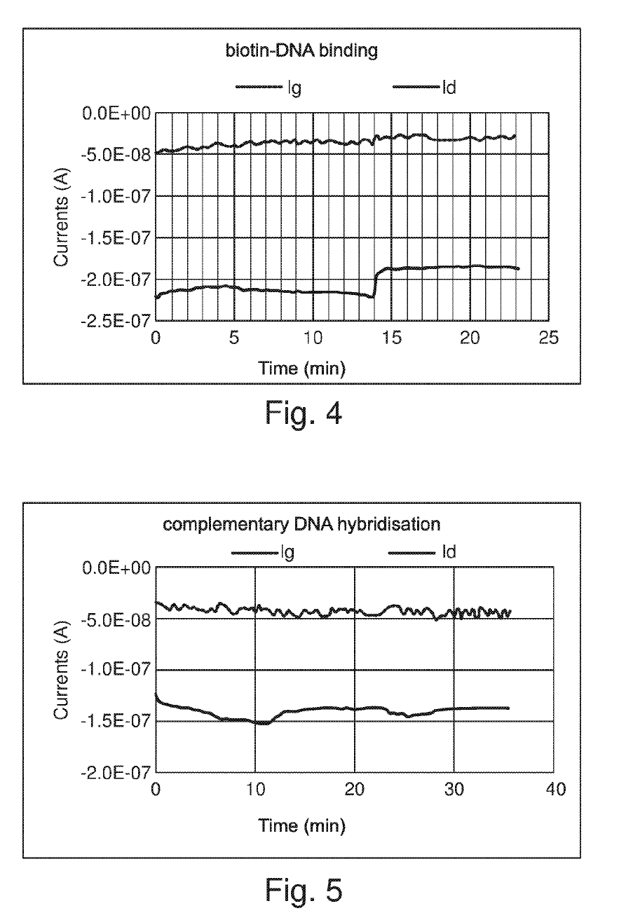

With reference to FIG. 5, a change in drain current is observed upon exposure of the sensor to an analyte solution comprising cDNA.

Although the present invention has been described in terms of specific exemplary embodiments, it will be appreciated that various modifications, alterations and/or combinations of features disclosed herein will be apparent to those skilled in the art without departing from the scope of the invention as set forth in the following claims.

1. A sensor comprising:
a bottom gate thin film transistor comprising a gate electrode; source and drain electrodes;
a dielectric layer between the gate electrode and the source and drain electrodes; and a semiconductor layer comprising an organic semiconducting material extending between the source and drain electrodes;
a binding layer comprising an amphiphilic polymer comprising a binding group adjacent to and in direct contact with the semiconductor layer; and
a receptor bound to the binding group.

2. A sensor according to claim 1 wherein the receptor is bound directly to the binding group.

3. A sensor according to claim 1 wherein the receptor is indirectly bound to the binding group.

4. A sensor according to claim 3 wherein the receptor and the binding group are bound to a linking group.

5. A sensor according to claim 4 wherein the linking group is streptavidin.

6. A sensor according to claim 1 wherein the amphiphilic polymer comprising the binding group is a protein comprising the binding group.

7. A sensor according to claim 6 wherein the protein is bovine serum albumin.

8. A sensor according to claim 1 wherein the binding group is selected from biotin and maleimide.

9. A sensor according to claim 1 wherein the receptor is selected from peptides, carbohydrates, antibodies, antigens, enzymes, proteins, cell receptors, DNA, RNA, PNA and aptamers.

10. A method of forming a sensor according claim 1, the method comprising the step of depositing a solution or suspension comprising the amphiphilic polymer onto the semiconductor layer.

11. A method according to claim 10, the method comprising the step of contacting the binding layer with a solution or suspension comprising the receptor.

12. A sensor comprising:
a bottom gate thin film transistor comprising a gate electrode; source and drain electrodes;
a dielectric layer between the gate electrode and the source and drain electrodes; and a semiconductor layer comprising an organic semiconducting material extending between the source and drain electrodes;
a binding layer comprising a protein comprising a binding material adjacent to and in direct contact with the semiconductor layer; and
a receptor bound to the binding group.

13. A method for detecting a target analyte, the method comprising the step of contacting a sensor according to claim 1 with an analyte fluid and measuring a response from the sensor.
